(19) United States
(12) Patent Application Publication
(10) Pub. No.: US 2026/0269077 A1
(43) Pub. Date: Sep. 10, 2026

(54) GENERATIVE DEFORMABLE TRANSFORMER FOR LONGITUDINAL CLINICAL ASSESSMENT

(71) Applicant: Genentech, Inc., South San Francisco, CA (US)

(72) Inventors: Mohammadreza NEGAHDAR, San Jose, CA (US); Joshua Mark GALANTER, Burlingame, CA (US); Degan HAO, Pittsburgh, PA (US)

(21) Appl. No.: 19/664,255

(22) Filed: Apr. 30, 2026

Related U.S. Application Data
(63) Continuation of application No. PCT/US2024/053576, filed on Oct. 30, 2024.
(60) Provisional application No. 63/594,183, filed on Oct. 30, 2023.
(60) Provisional application No. 63/594,790, filed on Oct. 31, 2023.

(51) Int. Cl. G16H 50/30; G06N 3/0455; G06N 3/08; G16H 30/40

(52) U.S. Cl. CPC G16H 50/30; G06N 3/0455; G06N 3/08; G16H 30/40

(57) ABSTRACT

A generative transformer model may be trained to generate, based on a first training image from a first timepoint, a first synthetic latent representation of a second training image that enables the second training image to be reconstructed therefrom. The generative transformer model may be trained to generate the first synthetic latent representation by shifting feature extraction to one or more regions in the first training image more likely to exhibit clinically significant changes between the first training image and the second training image. The trained generative transformer model may be applied generate, based on an input medical image, a second synthetic latent representation for a different timepoint than a timepoint of the input medical image. A clinical assessment computation model may be applied to determine, based on the second synthetic latent representation, a clinical assessment for the different timepoint in the absence of medical images from the different timepoint.

### CROSS REFERENCE TO RELATED APPLICATION

This application claims priority to U.S. Provisional Application No. 63/594,183, entitled “GENERATIVE DEFORMABLE TRANSFORMER FOR LONGITUDINAL CLINICAL ASSESSMENT” and filed on Oct. 30, 2023, and U.S. Provisional Application No. 63/594,790, entitled “GENERATIVE DEFORMABLE TRANSFORMER FOR LONGITUDINAL CLINICAL ASSESSMENT” and filed on Oct. 31, 2023, the disclosures of which are incorporated herein by reference in their entireties.

### TECHNICAL FIELD

The subject matter described herein relates generally to machine learning and more specifically to a machine learning based techniques for determining future clinical assessments based on presently available medical images.

### INTRODUCTION

Longitudinal analysis of medical images may provide insights into the progression of diseases over time. For example, two or more medical images, such as computed tomography (CT) scans and positron emission tomography (PET) scans, from different timepoints may be evaluated to identify indicators of metastatic disease (e.g., presence new lesions), stable disease (e.g., lesions that are neither increasing or decreasing in volume), and complete remission (e.g., absence of lesions). Given the dynamic nature of many diseases, such as cancers and neurodegenerative disorders (e.g., Alzheimer's disease, Parkinson's disease, and/or the like), tracking changes between medical images from multiple timepoints is often more clinically productive than examining medical images from single timepoints alone.

### SUMMARY

Systems, methods, and articles of manufacture, including computer program products, are provided for machine learning enabled longitudinal clinical assessment. In one aspect, there is provided a system for machine learning enabled longitudinal clinical assessment that includes at least one memory and at least one data processor. The at least one memory may store instructions that result in operations when executed by the at least one processor. The operations may include: training a generative transformer model to generate, based at least on a first training image from a first timepoint, a first synthetic latent representation of a second training image from a second timepoint that enables the second training image to be reconstructed from the first synthetic latent representation; applying the trained generative transformer model to generate, based at least on an input medical image, a second synthetic latent representation for a different timepoint than that of the input medical image; and determining, based at least on the second synthetic latent representation, a clinical assessment for the different timepoint.

In another aspect, there is provided a computer-implemented method for machine learning enabled longitudinal clinical assessment. The method may include: training a generative transformer model to generate, based at least on a first training image from a first timepoint, a first synthetic latent representation of a second training image from a second timepoint that enables the second training image to be reconstructed from the first synthetic latent representation; applying the trained generative transformer model to generate, based at least on an input medical image, a second synthetic latent representation for a different timepoint than that of the input medical image; and determining, based at least on the second synthetic latent representation, a clinical assessment for the different timepoint.

In another aspect, there is provided a computer program product including a non-transitory computer readable medium storing instructions. The instructions may cause operations may executed by at least one data processor. The operations may include: training a generative transformer model to generate, based at least on a first training image from a first timepoint, a first synthetic latent representation of a second training image from a second timepoint that enables the second training image to be reconstructed from the first synthetic latent representation; applying the trained generative transformer model to generate, based at least on an input medical image, a second synthetic latent representation for a different timepoint than that of the input medical image; and determining, based at least on the second synthetic latent representation, a clinical assessment for the different timepoint.

In some variations, one or more features disclosed herein including the following features can optionally be included in any feasible combination.

In some variations, the generative transformer model includes an encoder, a decoder, and an attention mechanism.

In some variations, the encoder is trained to generate a latent representation of a medical image by at least extracting, from the medical image, one or more latent features. The decoder is trained to reconstruct the medical image from the one or more latent features.

In some variations, the attention mechanism is a longitudinal deformable attention mechanism having a flexible range of self-attention across a plurality of pixels comprising a medical image.

In some variations, the attention mechanism includes a neural network.

In some variations, the training of the generative transformer model includes training the encoder to generate the first latent representation of the first training image that enables the decoder to reconstruct, from the first latent representation, the first training image.

In some variations, the training of the generative transformer model further includes training the attention mechanism to determine, based at least on the first latent representation of the first training image, one or more offsets to shift feature extraction to one or more regions of the first training image that differentiate the first training image from the second training image, and generate, based at least on the one or more regions of the first training image, the first synthetic latent representation of the second training image.

In some variations, the training of the generative transformer model further includes training the attention mechanism to generate the first synthetic latent representation of the second training image to enable the decoder to reconstruct, from the first synthetic latent representation, the second training image.

In some variations, the training of the generative transformer model includes applying the encoder to generate a latent representation of the second training image, and reducing a loss associated with a difference between the first synthetic latent representation of the second training image and the latent representation of the second training image.

In some variations, the training of the generative transformer model includes applying the decoder to reconstruct the second training image from the first synthetic latent representation of the second training image, and reducing a loss associated with a difference between the second training image and the second training image reconstructed from the first synthetic latent representation of the second training image.

In some variations, the attention mechanism includes a convolutional neural network.

In some variations, the attention mechanism includes a plurality of weight matrices. The training of the generative transformer model includes adjusting one or more weights included in the plurality of weight matrices.

In some variations, the plurality of weight matrices include a query matrix representative of a focus patch, a key matrix creating a plurality of key vectors measuring a relevance or similarity between the focus patch and other patches in the input medical image, and a value matrix generating value vectors comprising contextual information for each patch associated with the input medical image.

In some variations, the training of the generative transformer model includes determining, based at least on the query matrix, an offset to shift feature extraction to one or more regions of the first training image having a threshold likelihood of exhibiting a clinically significant change between the first timepoint of the first training image and the second timepoint of the second training image.

In some variations, the offset is applied to the key matrix and the value matrix in order to shift feature extraction to the one or more regions of the first training image having a threshold likelihood of exhibiting a clinically significant change between the first timepoint of the first training image and the second timepoint of the second training image.

In some variations, a clinical assessment computation model is applied to determine, based at least on the second synthetic latent representation, the clinical assessment for the different timepoint of the second synthetic latent representation.

In some variations, the clinical assessment computation model is trained based on training data that includes the second synthetic representation of the second training image. The training of the clinical assessment computation model includes applying the clinical assessment computation model to determine, based at least on the second synthetic representation, a clinical assessment for the second timepoint of the second training image.

In some variations, the training of the clinical assessment computation model further includes reducing a loss associated with a difference between the clinical assessment for the second timepoint of the second training image and a ground-truth clinical assessment for the second timepoint of the second training image.

In some variations, the clinical assessment computation model includes a feedforward neural network.

In some variations, the clinical assessment includes a risk for a disease developing or recurring at the different timepoint.

In some variations, the clinical assessment includes a progression of a disease at the different timepoint.

In some variations, the clinical assessment includes a survival prediction for a disease at the different timepoint.

In some variations, each of the first training image, the second training image, and the input medical image is a whole slide image (WSI), a computed tomography (CT) scan, a positron emission tomography (PET) scan, an X-ray, a magnetic resonance imaging (MRI) scan, and an ultrasound scan.

In some variations, the second synthetic latent representation corresponds to an unavailable medical image from the different timepoint. The trained generative transformer model generates the second synthetic latent representation absent the unavailable medical image.

In some variations, the different timepoint is prior to or subsequent to a timepoint of the input medical image.

In some variations, the input medical image is captured during patient screening or upon a relapse of a disease. The second latent representation corresponds to an unavailable medical image from a patient followup.

In some variations, the first synthetic latent representation includes one or more latent features extracted from one or more regions of the first training image more likely to exhibit changes between the first training image and the second training image.

In some variations, each latent feature of the one or more latent features comprise a hidden feature determined based on one or more observable features in the first training image.

In some variations, the one or more observable features include an intensity value of one or more pixels in the first training image.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the prediction of clinical outcomes in the context of medical imaging and risk prediction, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a longitudinal clinical assessment system, in accordance with some example embodiments;

FIG. 2 depicts a schematic diagram illustrating an example of an analysis engine, in accordance with some example embodiments;

FIG. 3 depicts a schematic diagram illustrating an example of a generative transformer model, in accordance with some example embodiments;

FIG. 4 depicts a flowchart illustrating an example process for machine learning enabled longitudinal clinical assessment, in accordance with some example embodiments;

FIG. 5A depicts a flowchart illustrating an example of a process for training a generative transformer model, in accordance with some example embodiments;

FIG. 5B depicts a flowchart illustrating an example of a process for training a clinical assessment computation model, in accordance with some example embodiments; and

FIG. 6 depicts a block diagram illustrating an example of a computing system, in accordance with some example embodiments.

When practical, similar reference numbers denote similar structures, features, or elements.

### DETAILED DESCRIPTION

Longitudinal analysis of medical images from multiple timepoints may provide more fruitful insights for assessing disease prognosis and treatment response than evaluating medical images from a single timepoint. For example, longitudinal analysis of medical images, such as whole slide images (WSI), computed tomography (CT) scans, positron emission tomography (PET) scans, X-rays, magnetic resonance imaging (MRI) scans, and ultrasound scans, may be performed to determine various clinical assessment for a patient. In some cases, these clinical assessment may include critical information for patient care including, for example, the risk of a disease (e.g., dynamic diseases such as cancer, neurodegenerative disorder, and/or the like) occurring, recurring, metastasizing, responding to a treatment, resolving, and/or the like.

Nevertheless, due to obstacles such as cost and accessibility, medical images from multiple timepoints may not always available. For example, in cases where some medical images from a present (or past) timepoint (e.g., baseline medical images captured during initial patient screening or upon a relapse of a disease) are available, no medical images may be available for a future timepoint (e.g., followup medical images captured during subsequent patient followup) to render a clinical assessment for the future timepoint. In some cases, medical images from either a past timepoint or the present timepoint may be available but in the absence of medical images from both timepoints, an accurate clinical assessment for the present timepoint cannot be made. For instance, where one or more medical images from a present timepoint are available but not medical images from a future timepoint, an accurate clinical assessment, such as risk prediction, cannot be rendered for the future timepoint. As such, in some cases, medical images from one timepoint may be used to determine a clinical assessment for a different timepoint for which medical images are unavailable. In particular, the present disclosure describes various machine learning based techniques in which medical images from a first timepoint may be used by a generative model to synthesize latent features of medical images from a second timepoint such that a clinical assessment for the second timepoint may be determined based on the synthesized latent features, thus obviating the need for medical images from the second timepoint.

In some example embodiments, an analysis engine may determine, based at least on an input medical image from a first timepoint, a clinical assessment for a second timepoint. The input medical image may be of a variety of modalities including, for example, a whole slide image (WSI), a computed tomography (CT) scan, a positron emission tomography (PET) scan, an X-ray, a magnetic resonance imaging (MRI) scan, an ultrasound scan, and/or the like. Moreover, in some cases, one or more medical images from the second timepoint may be unavailable due to a variety of reasons. As such, in some cases, the analysis engine may include a generative transformer model trained to generate, based at least on the input medical image from the first timepoint, a synthetic latent representation of an unavailable medical image that includes one or more synthetic latent features of the unavailable medical image. Moreover, the analysis engine may include a clinical assessment computation model (e.g., a feedforward neural network such as a multilayer perceptron (MLP) and/or the like) that determines, based at least on the synthetic latent representation of the unavailable medical image, a clinical assessment for the second timepoint. As described in more detail below, the clinical assessment for the second timepoint may be determined in the absence of medical images from the second timepoint. Instead, the generative transformer model may be trained to learn the changes that occur between medical images from different timepoints, thus enabling the generative transformer model to generate, based on the input medical image from the first timepoint but not any medical images from the second timepoint, the synthetic latent representation of those unavailable medical images.

In some example embodiments, the generative transformer model may include an encoder, a decoder, and an attention mechanism. In some cases, the encoder may embed the input medical image by at least extracting one or more latent features from the input medical image before the attention mechanism generates the synthetic latent representation of the unavailable medical image therefrom. It should be appreciated that a latent feature may be a hidden feature that is not directly observable but are instead determined based on one or more observable features. For example, an observable feature of the input medical image may include the intensity value of one or more pixels (or voxels) in the input medical image while a latent feature (or hidden feature) of the input medical image may be determined thereupon. As described in more detail below, the synthetic latent representation of the unavailable medical image from the second timepoint may include one or more latent features from one or more regions in the input medical image identified by the attention mechanism as containing features relevant to the clinical assessment at the second timepoint.

In some example embodiments, the generative transformer model may be trained based on one or more pairs of medical images from different timepoints such as, for example, a first training image from a first timepoint and a second training image from a second timepoint. For example, in some cases, the training of the generative transformer model may include training the encoder to generate, for the first training image, a first latent representation that enables the decoder to reconstruct the first training image therefrom. Furthermore, the training of the generative transformer model may include training the attention mechanism to generate, based at least on the first latent representation of the first training image, a synthetic latent representation of the second training image that enables the decoder to reconstruct the second training image therefrom. In some cases, the training of the generative transformer model may include adjusting the generative transformer model (e.g., one or more parameters of the generative transformer model) to reduce or minimize a loss function. The loss function may include a first loss associated with a first difference between the synthetic latent representation of the second training image and a second latent representation of the second training image generated, for example, by the encoder. Moreover, in some cases, the loss function may include a second loss associated with a second difference between the second training image and the second training image reconstructed, for example, by the decoder, from the synthetic latent representation of the second training image.

In some example embodiments, the attention mechanism may be a longitudinal deformable attention mechanism with a flexible attention range to shift attention to regions in a medical image more likely to exhibit or having a threshold likelihood of exhibiting clinically significant changes relative to another medical image from a different timepoint. As noted earlier, the attention mechanism may ingest the latent representation of the input medical image generated by the encoder. In some cases, as a longitudinal deformable attention mechanism, the attention mechanism may be trained to learn the regions in the input medical image that are more likely to exhibit or having a threshold likelihood of exhibiting clinically significant changes between the input medical image from the first timepoint and the unavailable medical image from the second timepoint. For instance, in some cases, the input medical image may be a baseline medical image captured during an initial patient screening or due to the relapse of a disease while the unavailable medical image may be a followup medical image captured during subsequent patient followup. While medical images from both timepoints may be available as part of the training data for training the generative transformer model, medical image from a future or past timepoint may be unavailable at inference time when the generative transformer model is deployed to operate on the input medical image. Accordingly, during training of the generative transformer model, the attention mechanism may be trained to shift feature extraction to regions in the input medical image exhibiting signs of malignancy that will develop by the second timepoint. For example, the attention mechanism may output, as part of the synthetic latent representation of the unavailable medical image from the second timepoint, the latent features corresponding to the regions in the input medical image more likely to exhibit or having a threshold likelihood of exhibiting clinically significant changes between the input medical image and the unavailable medical image.

As noted, the clinical assessment computation model may determine, based at least on the latent features forming the synthetic latent representation of the unavailable medical image, a clinical assessment for the second timepoint. That the synthetic latent representation of the unavailable medical image is generated to include latent features of regions in the input medical image exhibiting signs of malignancy that will develop by the second timepoint means that the clinical assessment computation model is able to render an accurate clinical assessment for the second timepoint even in the absence of the medical images from the second timepoint. It should be appreciated that the clinical assessment computation model may operate on the synthetic latent representation of the unavailable medical image instead of the synthesized version of the unavailable medical image itself, which could be generated by the decoder reconstructing the unavailable medical image from the synthetic latent representation of the unavailable medical image. Doing so may reduce the computational burden imposed by the generative transformer model by at least obviating the need for reconstructing the unavailable medical image and ensuring that the synthetic version of the unavailable medical image reconstructed from the synthetic latent representation bears sufficient visual similarities to the actual unavailable medical image in every anatomical region. That the clinical assessment computation model is able to render a clinical assessment based on the synthetic representation of the unavailable medical image (instead of the synthesized version thereof) also eliminates the computational resources required to register the input medical image and the unavailable medical image.

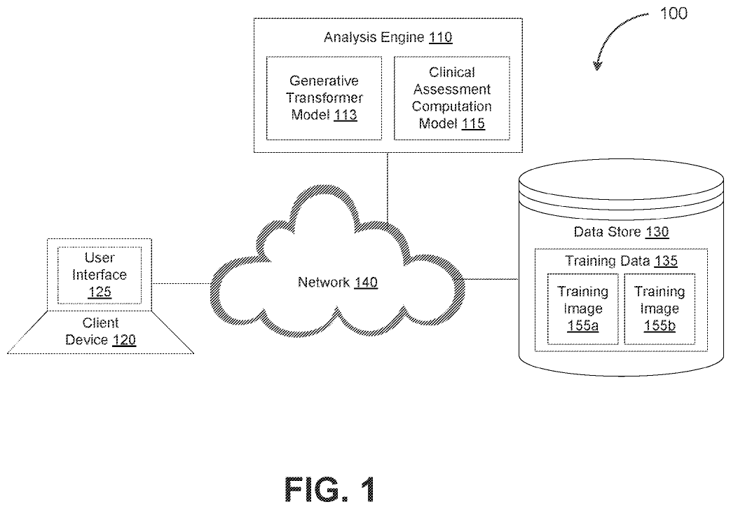

FIG. 1 depicts a system diagram illustrating an example of a longitudinal clinical assessment system 100, in accordance with some example embodiments. Referring to FIG. 1, the longitudinal clinical assessment system 100 may include an analysis engine 110, a client device 120 with a user interface 125, and a data store 130 storing training data 135. As shown in FIG. 1, the analysis engine 110, the client device 120, and the data store 130 may be communicatively coupled via a network 140. The client device 120 may be a processor-based device including, for example, a workstation, a desktop computer, a laptop computer, a smartphone, a tablet computer, a wearable apparatus, and/or the like. The data store 130 may be a relational database, a non-structured query language (NoSQL) database, an in-memory database, a graph database, a key-value store, a document store, and/or the like. The network 140 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the analysis engine 110 may perform longitudinal clinical assessment by at least determining, based at least on an input medical image from a first timepoint, a clinical assessment for a second timepoint in the absence of a medical image from the second timepoint. In some cases, the input medical image may be captured during an initial patient screening or upon a relapse of a disease (e.g., to rebaseline the patient). It should be appreciated that the interval (or the quantity of time) between the first timepoint and the second timepoint for which medical images are unavailable may vary depending on the circumstances. For example, for acute or critical illnesses, the interval between the first timepoint and the second timepoint may be as short as hours or days. Contrastingly, for condition that develops over longer time periods and chronic diseases, the interval between the first timepoint and the second timepoint may be more protracted and lasts months, years, and/or the like.

Referring again to FIG. 1, the analysis engine 110 may include a generative transformer model 113 and a clinical assessment computation model 115. In some cases, the generative transformer model 113 may be trained to generate, based at least on the input medical image from the first timepoint, a synthetic latent representation of an unavailable medical image from the second timepoint. The clinical assessment computation model 115 may determine, based at least on the synthetic latent representation of the unavailable medical image, a clinical assessment for the second timepoint. As described in more detail below, the generative transformer model 113 may generate the synthetic latent representation of the unavailable medical image by at least shifting feature extraction to one or more regions in the input medical image that are more likely to exhibit or having a threshold likelihood of exhibiting the changes between the input medical image from the first timepoint and the unavailable medical image from the second timepoint. It should be appreciated that the generative transformer model 113 may be trained to operate on two-dimensional images formed by pixels (e.g., a raster or a rectangular grid of pixels), each of which having an intensity value across one or more channels (e.g., a single channel for a grayscale image or three channels for a color image). In some cases, the generative transformer model 113 may also be trained to operate on three-dimensional volumes formed by a series of two-dimensional images. A three-dimensional volume may include multiple voxels, each of which corresponding to a pixel in one of the constituent two-dimensional images.

In some example embodiments, the generative transformer model 113 and the clinical assessment computation model 115 may be trained based on the training data 135. In some cases, the training data 135 may include pairs of medical images from different timepoints such as a first training image 155a from a first timepoint and a second training image 155b from a second timepoint. In some cases, the generative transformer model 113 may be trained in a self-supervised manner, meaning that the training of the generative transformer model 113 may be conducted without annotating the training data 135, such as the first training image 155a and the second training image 155b, with explicit ground-truth labels for the task of generating synthetic latent representations. Instead, the training of the generative transformer model 113 may include adjusting the generative transformer model 113 (e.g., one or more parameters of the generative transformer model 113) to reduce or minimize a loss function. As described in more detail below, the loss function may include a first loss associated with a first difference between a latent representation of the second training image 155b and a synthetic latent representation of the second training image 155b generated based on the first training image 155a. Moreover, in some cases, the loss function may include a second loss associated with a second difference between the second training image 155b and the second training image 155b reconstructed from the synthetic latent representation thereof.

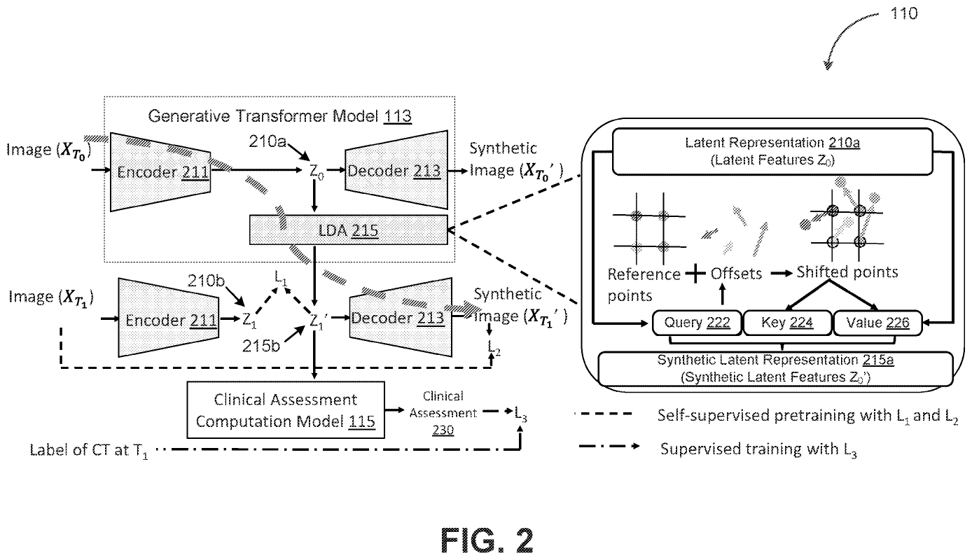

Contrastingly, in some cases, the clinical assessment computation model 115 may be trained in a supervised manner, meaning that the training of the clinical assessment computation model 115 may be conducted based on explicit ground-truth labels associated with the task of rendering clinical assessments. For example, in some cases, the training of the clinical assessment computation model 115 may include reducing or minimizing a loss function quantifying a difference between the clinical assessment determined by the clinical assessment computation model 115 based on the synthetic latent representation of a medical image and the ground-truth clinical assessment associated with the medical image. The training of the generative transformer model 113 and the clinical assessment model 115 is further shown in FIG. 2.

Referring now to FIG. 2, the generative transformer model 113 may include an encoder 211 (e.g., a convolutional encoder) and a decoder 213 (e.g., a transposed convolutional decoder) that, in some cases, form an autoencoder architecture. Furthermore, as shown in FIG. 2, the generative transformer model 113 may include an attention mechanism 215 which, in some cases, may be a longitudinal deformable attention (LDA) mechanism. In some example embodiments, the generative transformer model 113 may be trained based on one or more pairs of medical images from different timepoints. For example, as shown in FIG. 2, the generative transformer model 113 may be trained based on a first image X_{T}_{₀} from a first timepoint T₀ and a second image X_{T}_{₁} from a second timepoint T₁. It should be appreciated that in the context of FIG. 2, the first image X_{T}_{₀} from the first timepoint T₀ and the second image X_{T}_{₁} from the second timepoint T₁ are a pair of training images. While both the first image X_{T}_{₀} from the first timepoint T₀ and the second image X_{T}_{₁} from the second timepoint T₁ may be available for training the generative transformer model 113, either the first image X_{T}_{₀} from the first timepoint T₀ or the second image X_{T}_{₁} from the second timepoint T₁ may be unavailable at inference time when the trained generative transformer model 113 is deployed in conjunction with the clinical assessment computation model 115. For instance, where the second image X_{T}_{₁} from the second timepoint T₁ is unavailable at inference time, the trained generative transformer model 113 may generate a synthetic latent representation of the second image X_{T}_{₁} that enables the clinical assessment computation model 115 to generate a clinical assessment 230 for the second timepoint T₁.

In some cases, the first image X_{T}_{₀} and the second image X_{T}_{₁} may be medical images of a variety of different modalities including, for example, a whole slide image (WSI), a computed tomography (CT) scan, a positron emission tomography (PET) scan, an X-ray, a magnetic resonance imaging (MRI) scan, an ultrasound scan, and/or the like. Moreover, while the first image X_{T}_{₀} and the second image X_{T}_{₁} may be two-dimensional images or three-dimensional volumes formed by a series of two-dimensional images, the generative transformer model 113 may operate on one-dimensional sequence of token embeddings corresponding to each of the first image X_{T}_{₀} and the second image X_{T}_{₁}. As such, in some cases, the first image X_{T}_{₀} and the second image X_{T}_{₁} may undergo preprocessing prior to being ingested by the generative transformer model 113. In some cases, this preprocessing may include transforming the raster of pixels in each of the first image X_{T}_{₀} and the second image X_{T}_{₁} into a sequence of flattened two-dimensional patches. For instance, in some cases, the preprocessing of the first image X_{T}_{₀} and the second image X_{T}_{₁} may include one or more of resizing, patch embedding (e.g., to split each image into fixed-size patches and generate a sequence of patches therefrom), positional embedding (e.g., to provide a relative position of each pixel (or voxel) in the corresponding image), patch embedding (e.g., to capture the relative positions of each pixel in the individual patches), and/or the like.

In some cases, the training of the generative transformer model 113 may include training the encoder 211 to generate a first latent representation 210a (including a first set of latent features Z₀) of the first image X_{T}_{₀} that enables the decoder 213 to generate therefrom a first synthetic image X_{T}_{₀}′ that reconstructs the first image X_{T}_{₀} with minimal loss (or maximum fidelity) relative to the first image X_{T}_{₀}. Moreover, the training of the generative transformer model 113 may include training the encoder 211 to generate a second latent representation 210b (including a second set of latent features Z₁) of the second image X_{T}_{₁}, which may be used to determine at least a portion of the loss associated with the generative transformer model 113.

In some example embodiments, the training of the generative transformer model 113 may further include training the attention mechanism 215 to generate, based at least on the first latent representation 210a (including the first set of latent features Z₀) of the first image X_{T}_{₀}, a synthetic latent representation 215b (including a set of synthetic latent features Z₁′) of the second image X_{T}_{₁} from the second timepoint T₁. In some cases, the attention mechanism 215 may be trained to generate the synthetic latent representation 215b (including the set of synthetic latent features Z₁′) of the second image X_{T}_{₁} from the second timepoint T₁ by at least learning which regions in the first image X_{T}_{₀} are more likely to exhibit or having a threshold likelihood of exhibiting clinically significant changes between the first image X_{T}_{₀} and the second image X_{T}_{₁}. In doing so, the attention mechanism 215 may generate the synthetic latent representation 215b (including the set of synthetic latent features Z₁′) to encapsulate changes between the first timepoint T₀ and the second timepoint T₁. As explained in more detail below, in instances where the attention mechanism 215 is implemented as a longitudinal deformable attention mechanism (LDA), the attention mechanism 215 may determine one or more offsets to shift feature extraction to the one or more regions in the first image X_{T}_{₀} more likely to exhibit or having a threshold likelihood of exhibiting clinically significant changes between the first image X_{T}_{₀} and the second image X_{T}_{₁}.

In the context of image processing, the attention mechanism 215 may operate on patches (or groups of adjacent pixels or voxels) within the first image X_{T}_{₀} in order to generate the synthetic latent representation 215b (including the set of synthetic latent features Z₁′) of the second image X_{T}_{₁}. The parameters of the attention mechanism 215, which are adjusted during training, may include three weight matrices: a query matrix 222 representative of a focus patch in the first image X_{T}_{₀}, a key matrix 224 used to create key vectors measuring the relevance or similarity between the focus patch and other patches in the first image X_{T}_{₀}, and a value matrix 226 used to generate value vectors containing contextual information for individual patches in the first image X_{T}_{₀}. In other words, the query matrix 222 may lend focus to a patch of interest in the first image X_{T}_{₀}, the key matrix 224 may measures the relevance between different patches in the first image X_{T}_{₀}, and the value matrix 226 may provide the context for creating a final contextual representation of the focus patch. When combined, the three weight matrices 222, 224, and 226 may enable the attention mechanism 215 to capture the relationships and dependencies between different patches in the first image X_{T}_{₀}, including distant relationships or dependencies between patches that are not immediately adjacent to one and another.

As such, in some cases, the weight matrices 222, 224, and 226 may be applied to an input, such as the first image X_{T}_{₀}, in order to project the input into the query, key, and value vectors. Moreover, in some cases, the attention mechanism 215 may operate by at least mapping, for individual patches in the first image X_{T}_{₁}, the corresponding query, key, and value vectors to an output which, in this case, may be the synthetic latent representation 215b (including the set of synthetic latent features Z₁′) of the second image X_{T}_{₁} from the second timepoint T₁. In some cases, the output of the attention mechanism 215 may be a weighted sum of the values in which the weight assigned to each value is computed by evaluating a compatibility function (e.g., cosine similarity) between the query with the corresponding key.

In some example embodiments, the attention mechanism 215 may be a longitudinal deformable attention (LDA) mechanism having a flexible attention range to shift attention to regions in the first image X_{T}_{₀} more likely to exhibit or having a threshold likelihood of exhibiting clinically significant changes relative to the second image X_{T}_{₁}. The attention range of the attention mechanism 215 refers to the proportion of the input considered by the attention mechanism 215 when determining the relationship or dependencies between the constituent elements. In the case of image processing, which operates on patches (or groups of adjacent pixels) within the first image X_{T}_{₀}, for example, the attention range of the attention mechanism 215 may correspond to the proportion of patches in the first image X_{T}_{₀} considered by the attention mechanism 215 when determining the relationship or dependencies between the individual patches of the first image X_{T}_{₀}. As a conventional attention mechanism, the attention range of the attention mechanism 215 may be fixed, in some cases, to every patch in the first image X_{T}_{₀}. However, when implemented as a longitudinal deformable attention mechanism, the attention range of the attention mechanism 215 may vary, depending on the first image X_{T}_{₀}, to encompass different subsets of patches within the first image X_{T}_{₀}. Where the attention range of the attention mechanism 215 is limited to a subset of patches in the first image X_{T}_{₀}, the attention mechanism 215 may determine the relationship or dependencies between each patch in the subset of patches.

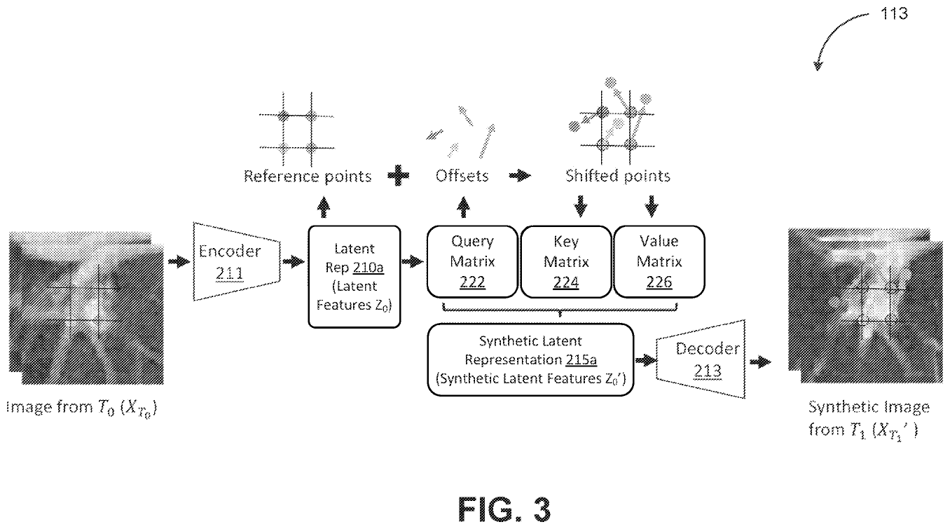

In some example embodiments, as a longitudinal deformable attention (LDA) mechanism, the attention range of the attention mechanism 125 may be adjusted based on the first image X_{T}_{₀} when shifting feature extraction therefrom in order to generate the synthetic latent representation 215b (including the set of synthetic latent features Z₁′) to capture the changes that are present between the first image X_{T}_{₀} and the second image X_{T}_{₁}. This may be accomplished at least in part by the attention mechanism 125 shifting attention to the regions of the first image X_{T}_{₀} more likely to exhibit or having a threshold likelihood of exhibiting clinically significant changes, such as the progression of a disease, between the first image X_{T}_{₀} and the second image X_{T}_{₁}. For instance, in the example shown in FIGS. 2-3, the attention mechanism 215 may be a convolutional neural network (CNN) that learns the offsets (denoted by the arrows in FIGS. 2-3) from the query matrix 222 before using these offsets to shift the key matrix 224 and the value matrix 226 to the regions in the first image X_{T}_{₀} more likely to exhibit or having a threshold likelihood of exhibiting clinically significant changes between the first timepoint T₀ of the first image X_{T}_{₀} and the second timepoint T₁ of the second image X_{T}_{₁}. That is, depending on the first image X_{T}_{₀}, the attention mechanism 215 may shift feature extraction from one or more reference point to one or more corresponding shifted points in the first image X_{T}_{₀}. In some cases, the set of synthetic latent features Z₁′ forming the synthetic latent representation 215b of the second image X_{T}_{₁} may include one or more latent features zs extracted from those regions in the first image X_{T}_{₀}.

In the example shown in FIG. 3, for instance, the attention mechanism 215 may highlight those regions in the first image X_{T}_{₀} more likely to exhibit signs of disease progression from the first timepoint T₀ and the second timepoint T₁ by at least learning, from the query matrix 222 focusing on one or more patches of interest in the first image X_{T}_{₀}, one or more offsets (denoted by the arrows) from one or more corresponding reference points in the first image X_{T}_{₀} before applying the offsets to the key matrix 224 and the value matrix 226 to shift feature extraction to those regions. Equation (1) below expresses the feature extraction shift performed by the attention mechanism 215.

$$Δ c = {proj}_{q} ( q ) \qquad ( 1 )$$

wherein Δc denotes the offset (or the shift in coordinates), q denotes the query, and proj_{q} denotes the machine learning model (e.g., a convolutional neural network and/or the like) computing the offsets Δc.

Equation (2) below expression the extraction of synthetic latent features zi from the regions in the first image X_{T}_{₀} more likely to exhibit signs of disease progression from the first timepoint T₀ and the second timepoint T₁.

$${z}_{1}^{′} = {proj}_{out} ( softmax ( \frac{q \cdot k}{\sqrt{{d}_{k}}} ) \cdot v ) \qquad ( 2 )$$

wherein k denote the key features (e.g., of size dₖ) and v denotes the value features. In Equation (2), projₒᵤₜ denotes a machine learning model (e.g., a convolutional neural network and/or the like) that converts attention features to the set of synthetic latent features Z₁′ that form the synthetic latent representation 215b of the second image X_{T}_{₁}.

The extraction of the key features k and the value features v in Equation (2) are expressed by Equations (3) and (4) below.

$$k = {proj}_{k} ( sample ( {z}_{0} , Δc ) ) \qquad ( 3 )$$

$$v = {proj}_{v} ( sample ( {z}_{0} , Δc ) ) \qquad ( 4 )$$

wherein projₖ denotes a machine learning model (e.g., a convolutional neural network and/or the like) that converts the latent features z₀ in the first image X_{T}_{₁} to key features k, and projᵥ denotes a machine learning model (e.g., a convolutional network and/or the like) that converts the latent features z₀ in the first image X_{T}_{₁} to value features v.

Referring again to FIG. 2, in some cases, the training of the generative transformer model 113 may be trained in a self-supervised manner, meaning that the generative transformer model 113 may be trained without explicit ground-truth labels for the task of generating the synthetic latent representation 215b (including the set of synthetic latent features Z₁′) of the second image X_{T}_{₁}. Instead, in the example shown in FIG. 2, the training of the generative transformer model 113 may be performed based on implicit labels, such as the second latent representation 210b (including the set of latent features Z₁) of the second image X_{T}_{₁} and the second synthetic image X_{T}_{₁} generated based on the synthetic latent representation 215b (including the set of synthetic latent features Z₁′) of the second image X_{T}_{₁}. For example, in some cases, the training of the generative transformer model 113 may include adjusting one or more parameters of the generative transformer model 113 to reduce or minimize a loss function L of the second image X_{T}_{₁} shown as Equation (5) below.

$$L = {λ}_{1} \cdot {L}_{1} ( {Z}_{1} , {Z}_{1}^{′} ) + {λ}_{2} \cdot {L}_{2} ( {Z}_{{T}_{1}} , {Z}_{{T}_{1}}^{′} ) \qquad ( 5 )$$

wherein L₁ denotes a first loss, L₂ denotes a second loss, λ₁ denotes the first weight assigned to the first loss L₁, and λ² denotes a second weight assigned to the second loss L₂.

As shown in Equation (5), the training of the generative transformer model 113 may include reducing or minimizing a first loss term L₁ corresponding to a first loss (e.g., mean squared error (MSE) and/or the like) between the second latent representation 210b (including the set of latent features Z₁) of the second image X_{T}_{₁} generated by the encoder 211 based on the second image X_{T}_{₁} and the synthetic latent representation 215b (including the set of synthetic latent features Z₁′) of the second image X_{T}_{₁} generated by the attention mechanism 215. Furthermore, Equation (5) shows that the training of the generative transformer model 113 may include reducing or minimizing a second loss term L₂ corresponding to a second loss (e.g., mean squared error (MSE) and/or the like) between the second image X_{T}_{₁} and a reconstruction of the second image X_{T}_{₁}′ generated by the decoder 213 based on the synthetic latent representation 215b (including the set of synthetic latent features Z₁′) of the second image X_{T}_{₁}. In some cases, reducing or minimizing the loss function L may include increasing or maximizing the similarity between the second latent representation 210b (including the set of latent features Z₁) of the second image X_{T}_{₁} generated by the encoder 211 based on the second image X_{T}_{₁} and the synthetic latent representation 215b (including the set of synthetic latent features Z₁′) of the second image X_{T}_{₁} generated by the attention mechanism 215. Moreover, in some cases, reducing or minimizing the loss function L may include increasing or maximizing the fidelity of the reconstruction of the second image X_{T}_{₁}′ generated based on the synthetic latent representation 215b (including the set of synthetic latent features Z₁′) of the second image X_{T}_{₁}.

In some example embodiments, the clinical assessment computation model 115 may ingest the synthetic latent representation 215b (including the set of synthetic latent features Z₁′) of the second image X_{T}_{₁} generated by the attention mechanism 215. For example, as shown in FIG. 2, the clinical assessment computation model 115 may determine, based at least on the synthetic latent representation 215b (including the set of synthetic latent features Z₁′) of the second image X_{T}_{₁}, a clinical assessment for the second timepoint without the second image X_{T}_{₁} from the second timepoint T₁. In some cases, the clinical assessment computation model 115 may be trained in a supervised manner, meaning that the clinical assessment computation model 115 may be trained with explicit ground-truth labels associated with the task of rendering clinical assessments. For instance, FIG. 2 shows that the training of the clinical assessment computation model 115 may output the clinical assessment 230, which may include an indication of a risk for a disease developing or recurring, a progression of the disease, and/or a survival prediction for the disease at the second timepoint T₁. The error present in the clinical assessment 230 is denoted by a third loss L₃, which corresponds to a third difference between the clinical assessment 230 made by the clinical assessment computation model 115 based on the synthetic latent representation 215b (including the set of synthetic latent features Z₁′) of the second image X_{T}_{₁} and a ground-truth clinical assessment associated with the second image X_{T}_{₁}. In some cases, the training of the clinical assessment computation model 115 may include adjusting one or more parameters (e.g., weights, biases, and/or the like) of the clinical assessment computation model 115 to reduce (or minimize) the third loss L₃.

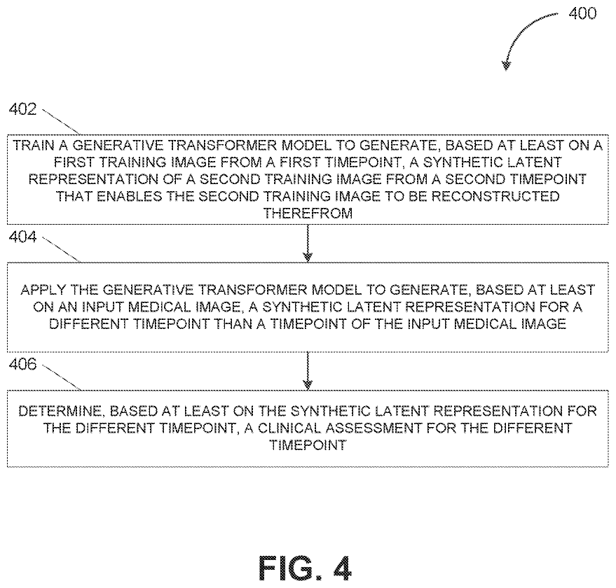

FIG. 4 depicts a flowchart illustrating an example of a process 400 for machine learning enabled longitudinal clinical assessment, in accordance with some example embodiments. Referring to FIGS. 1-4, the process 400 may be performed by the analysis engine 110 to train the generative transformer model 113 to generate, based at least on a first training image from a first timepoint, a synthetic latent representation of a second training image from a second timepoint such that a clinical assessment for the second timepoint may be rendered in the absence of the second training image from the second timepoint. For example, in some cases, the first training image from the first timepoint may be a baseline medical image captured at a present timepoint (e.g., as a part of initial patient screening or due to the relapse of a disease) while the second training image from the second timepoint may be a followup medical image captured during patient followup performed at a future timepoint. In some cases, the generative transformer model 113 may be trained to generate the synthetic latent representation of the second training image to include latent features of regions in the first training image exhibiting signs of malignancy that will develop by the second timepoint. Accordingly, once trained, the generative transformer model 113 may generate, based at least on the baseline medical image from the present timepoint, a synthetic latent representation of the followup medical image such that the clinical assessment computation model 115 is able to render a clinical assessment (e.g. risk prediction) for the future timepoint in the absence of the followup medical image from the future timepoint. In some cases, the clinical assessment computation model 115 may be trained to operate on the synthetic latent representation of the second training image instead of the synthesized version of the second training image itself, which eliminates the computational burden associated with reconstructing the second training image and registering the first training image and the second training image.

At 402, the analysis engine 110 may train the generative transformer model 113 to generate, based at least on a first training image from a first timepoint, a synthetic latent representation of a second training image that enables the second training image to be reconstructed therefrom. In some example embodiments, the analysis engine 110 may train the generative transformer model 113 to generate the synthetic latent representation 215b (including the set of synthetic latent features Z₁′) of the second training image X_{T}_{₁} from the second timepoint T₁ in the absence of the second training image X_{T}_{₁}. Instead, as shown in FIGS. 2-3, the generative transformer model 113 may be trained to generate, based at least on the first image X_{T}_{₀} from the first timepoint T₀, the synthetic latent representation 215b (including the set of synthetic latent features Z₁′) of the second image X_{T}_{₁} from the second timepoint T₁. As described in more detail below, the generative transformer model 113 may be trained to shift feature extraction to one or more regions in the first image X_{T}_{₀} more likely to exhibit or having a threshold likelihood of exhibiting clinically significant changes between the first image X_{T}_{₀} and the second image X_{T}_{₁}. For instance, in some cases, the generative transformer model 113 may be trained to learn regions in the first image X_{T}_{₀} more likely to exhibit signs of malignancy that will develop by the second timepoint T₁. Accordingly, the synthetic latent representation 215b (including the set of synthetic latent features Z₁′) may enable clinical assessment to be rendered for the second timepoint T₁ even when the second image X_{T}_{₁} for the second timepoint T₁ is unavailable.

At 404, the analysis engine 110 may apply the generative transformer model to generate, based at least on an input medical image, a synthetic latent representation for a different timepoint than a timepoint of the input medical image. In some cases, once trained, the analysis engine 110 may apply the generative transformer model 113 to generate, based at least on a medical image from one timepoint, a synthetic latent representation for a different timepoint in the absence of any medical images from that timepoint. For example, in some cases, the generative transformer model 113 may be applied to generate, based at least on an input image from one timepoint, a synthetic latent representation of an unavailable medical image from a different timepoint. In some cases, the input medical image may be from a present (or past) timepoint, such as a baseline medical image captured during initial patient screening or upon a relapse of a disease (e.g., to rebaseline the patient). Meanwhile, the unavailable medical image may be a followup medical image captured at a future timepoint, such as during one or more subsequent patient visits. Despite the absence of the unavailable medical image, the generative transformer model 113 may generate the synthetic latent representation to include one or more latent features extracted from one or more regions of the input medical image more likely to exhibit or having a threshold likelihood of exhibiting clinically significant changes, such as disease progression, between the two different timepoints. Thus, as described in more detail below, the synthetic latent representation may for the basis upon which the clinical assessment computation model 115 is able to render a clinical assessment for timepoint of the unavailable medical image.

At 406, the analysis engine 110 may determine, based at least on the synthetic latent representation for the different timepoint, a clinical assessment for the different timepoint. In some example embodiments, the analysis engine 110 may apply the clinical assessment computation model 115 to determine, based at least on the synthetic latent representation of the unavailable medical image for the different timepoint, a clinical assessment for the different timepoint (e.g., a past timepoint, a future timepoint, and/or the like). In some cases, the clinical assessment computation model 115 may be a machine learning model (e.g., a feedforward neural network such as a multilayer perceptron (MLP) and/or the like) trained to determine, based at least on the synthetic latent representation generated based on the medical image from one timepoint, a clinical assessment for the different timepoint even though no medical images from that timepoint may be available. For example, in some cases, the clinical assessment computation model 115 may be applied to determine, based at least on the synthetic latent representation of the unavailable medical image, a clinical assessment for the past or future timepoint of the unavailable medical image despite lacking access to the unavailable medical image. Examples of clinical assessments that may be rendered by the clinical assessment computation model 115 may include one or more of a risk of a disease developing, a risk of a disease recurring, a progression of a disease, a survival for a disease, and/or the like.

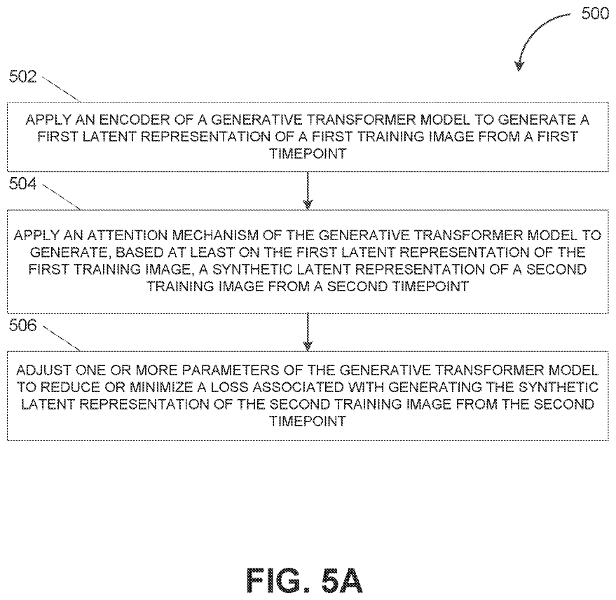

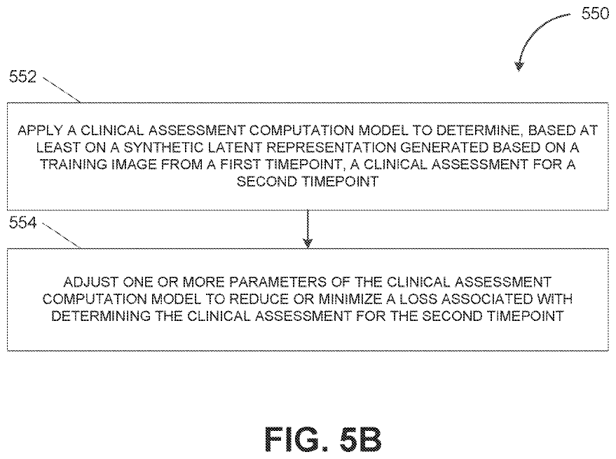

FIG. 5A depicts a flowchart illustrating an example of a process 500 for machine learning enabled longitudinal clinical assessment, in accordance with some example embodiments. Referring to FIGS. 1-4 and 5A, the process 500 may be performed by the analysis engine 110 to train the generative transformer model 113 to generate, based at least on a first training image from a first timepoint, a synthetic latent representation of a second training image from a second timepoint. The generative transformer model 113 may be trained to generate the synthetic latent representation to include latent features of regions in the first training image exhibiting signs of malignancy that will develop by the second timepoint. As such, in some cases, the clinical assessment computation model 115 may be able to render an accurate clinical assessment for the second timepoint even in the absence of medical images from the second timepoint. Moreover, the clinical assessment computation model 115 may operate on the synthetic latent representation of the second training image instead of the synthesized version of the second training image itself, thereby reducing the computational burden associated with reconstructing the second training image. That the clinical assessment computation model is able to render a clinical assessment based on the synthetic latent representation of the second training image (instead of the synthesized version thereof) also eliminates the computational resources required to register the first training image and the second training image.

In some cases, the process 500 may implement operation 402 of the process 400 shown in FIG. 4. For example, in some cases, the first training image from the first timepoint may be a baseline medical image from the present timepoint that is captured as part of an initial patient screening or due to the relapse of a disease while the second training image is a followup medical image captured during a subsequent patient followup that is performed at a future timepoint. In some cases, the process 500 may be performed to train the generative transformer model 113 to generate, based on an input medical image from one timepoint, a synthetic latent representation for a different timepoint for which medical images are unavailable. Doing so may enable the rendering of a longitudinal clinical assessment across multiple timepoints even though medical images are available for a single timepoint.

At 502, the analysis engine 110 may apply the encoder 211 of the generative transformer model 113 to generate a first latent representation of a first training image from a first timepoint. For example, as shown in FIG. 2, the training of the generative transformer model 113 may include training the encoder 211 to generate the first latent representation 210a (including the first set of latent features Z₀) of the first training image X_{T}_{₀} to enable the decoder 213 of the generative transformer model 113 to reconstruct the first training image X_{T}_{₀} therefrom.

At 504, the analysis engine 110 may apply the attention mechanism 215 of the generative transformer model 113 to generate, based at least on the first latent representation of the first training image, a synthetic latent representation of a second training image from a second timepoint. For example, in some cases, the analysis engine 110 may train the attention mechanism 215 to generate, based at least on the first latent representation 210a (including the first set of latent features Z₀) of the first image X_{T}_{₀}, the synthetic latent representation 215b (including the set of synthetic latent features Z₁′) of the second image X_{T}_{₁} from the second timepoint T₁. As noted, the attention mechanism 215 may be trained to shift feature extraction to those regions in the first image X_{T}_{₀} more likely to exhibit or having a threshold likelihood of exhibiting clinically significant changes between the first image X_{T}_{₀} and the second image X_{T}_{₁}. For instance, in the examples shown in FIGS. 2-3, the attention mechanism 215 may be trained to determine one or more offsets to shift feature extraction to the one or more regions in the first image X_{T}_{₀} more likely to exhibit or having a threshold likelihood of exhibiting clinically significant changes between the first image X_{T}_{₀} and the second image X_{T}_{₁}. In some cases, the attention mechanism 215 may be a convolutional neural network (CNN) that learns the offsets (denoted by the arrows in FIGS. 2-3) from the query matrix 222 before using these offsets to shift the key matrix 224 and the value matrix 226 to the regions in the first image X_{T}_{₀} more likely to exhibit or having a threshold likelihood of exhibiting clinically significant changes between the first image X_{T}_{₀} and the second image X_{T}_{₁}. In some cases, the synthetic latent representation 215b (including the set of synthetic latent features Z₁′) of the second image X_{T}_{₁} output by the attention mechanism 215 may include one or more latent features extracted from those regions in the first image X_{T}_{₀} more likely to exhibit or having a threshold likelihood of exhibiting clinically significant changes between the first image X_{T}_{₀} and the second image X_{T}_{₁}.

At 506, the analysis engine 110 may adjust one or more parameters of the generative transformer model 113 to reduce or minimize a loss associated with generating the synthetic latent representation of the second training image from the second timepoint. In some example embodiments, the training of the generative transformer model 113 may include adjusting one or more parameters of the generative transformer model 113 to reduce or minimize the losses (or errors) associated with the task of generating the synthetic latent representation 215b (including the set of synthetic latent features Z₁′) of the second image X_{T}_{₁}. In some cases, the generative transformer model 113 may be trained in a self-supervised manner, meaning that the generative transformer model 113 may be trained without explicit ground-truth labels for the task of generating the synthetic latent representation 215b (including the set of synthetic latent features Z₁′) of the second image X_{T}_{₁}. Moreover, in some cases, the training of the generative transformer model 113 may include adjusting one or more parameters of the generative transformer model 113 to reduce the loss function shown as Equation (5) above. For example, in some cases, the training of the generative transformer model 113 may include adjusting one or more parameters of the generative transformer model 113 to reduce or minimize a first loss (e.g., the first loss term L₁ in Equation (5)) between the second latent representation 210a (including the second set of latent features Z₁) of the second image X_{T}_{₁} generated by the encoder 211 based on the second image X_{T}_{₁} and the synthetic latent representation 215 (including the set of synthetic latent features Z₁′) of the second image X_{T}_{₁} generated by the attention mechanism 215. Furthermore, the training of the generative transformer model 113 may include adjusting one or more parameters of the generative transformer model 113 to reduce or minimize a second loss (e.g., the second loss term L₂) between the second image X_{T}_{₁} and a reconstruction of the second image X_{T}_{₁}′ generated by the decoder 213 based on the synthetic latent representation 215b (including the set of synthetic latent features Z₁′) of the second image X_{T}_{₁}.

FIG. 5B depicts a flowchart illustrating an example of a process 550 for machine learning enabled longitudinal clinical assessment, in accordance with some example embodiments. Referring to FIGS. 1-4 and 5B, the process 550 may be performed by the analysis engine 110 to train the clinical assessment computation model 115 to determine, based at least on a synthetic latent representation generated based on a medical image from one timepoint, a clinical assessment for a different timepoint without any medical images from that timepoint. In some cases, the process 550 may be performed to train the clinical assessment computation model 115 for use in performing operation 406 of the process 400 shown in FIG. 4. As noted, the generative transformer model 113 may be trained to generate the synthetic latent representation to include latent features of regions in the first training image exhibiting signs of malignancy that will develop by the second timepoint. Accordingly, the clinical assessment computation model 115 may be trained to render an accurate clinical assessment for the second timepoint without the second training image from the second timepoint. Furthermore, the clinical assessment computation model 115 may be trained to operate on the synthetic latent representation of the second training image instead of the synthesized version of the second training image itself. Doing so may eliminate the computational burdens associated with reconstructing the second training image and that of registering the first training image and the second training image.

At 552, the analysis engine 110 may apply the clinical assessment computation model 115 to determine, based at least on a synthetic latent representation generated based on a training image from a first timepoint, a clinical assessment for a second timepoint. For instance, in the example shown in FIG. 2, the clinical assessment computation model 115 may be applied to determine, based at least on the synthetic latent representation 215b (including the set of synthetic latent features Z₁′) of the second image X_{T}_{₁}, a clinical assessment for the second timepoint T₁ in the absence of the second image X_{T}_{₁} itself. That is, the synthetic latent representation 215b (including the set of synthetic latent features Z₁′) of the second image X_{T}_{₁} may be generated, for example, by the generative transformer model 113, based on the first image X_{T}_{₀}. In particular, as noted, the generative transformer model 113 may be trained to generate the synthetic latent representation 215b to include one or more latent features Z₁′ extracted from regions of the first image X_{T}_{₀} more likely to exhibit clinically significant changes, such as disease progression, between the first image X_{T}_{₀} and the second image X_{T}_{₁}.

At 554, the analysis engine 110 may adjust one or more parameters of the clinical assessment computation model 115 to reduce or minimize a loss associated with determining the clinical assessment for the second timepoint. In some example embodiments, the training of the clinical assessment computation model 115 may be performed in a supervised manner, meaning that the clinical assessment computation model 115 may be trained with explicit ground-truth labels for the task of rendering a clinical assessment. Accordingly, as shown in FIG. 2, the training of the clinical assessment computation model 115 may include adjusting one or more parameters of the clinical assessment computation model 115 (e.g., one or more weights of the feed forward neural network) to reduce or minimize a loss (e.g., the third loss L₃) between the clinical assessment made by the clinical assessment computation model 115 based on the synthetic latent representation 215b (including the set of synthetic latent features Z₁′) of the second image X_{T}_{₁} and a ground-truth clinical assessment associated with the second image X_{T}_{₁}. Doing so may ensure that when the clinical assessment computation model 115 is deployed to operate on actual data, the clinical assessment computation model 115 is able to generate, based on the synthetic latent representation for a future or past timepoint, an accurate clinical assessment despite not having access to any medical images from that timepoint.

### Example Experiments

The performance of the analysis engine 110 including the generative transformer model 113 and the clinical assessment computation model 115 was validated with experiments on a first dataset from the national lung screening trial (NLST) and a second dataset from the open-source imaging consortium (OSIC).

The first experiment was conducted on the first dataset, which includes 5,511 patients selected, based at least on the availability of computed tomography (CT) scans from multiple timepoints (i.e., three timepoints within three years), from a total of 26,722 patients in the national lung screening trial (NLST) who were at high risk of lung cancer and enrolled in the screening with low-dose computed topography (CT) scans. The analysis engine 110 was deployed to perform the task of predicting, for each patient, the risk of lung cancer in the third year using computed tomography (CT) scans from the first year and second year.

In the second experiment, the analysis engine 110 was deployed to predict patient survival based on computed tomography (CT) scans from two different timepoints. These computed tomography (CT) scans originate from the open-source imaging consortium (OSIC) and include patients with pulmonary fibrosis and lung function measurement through forced vital capacity (FVC). Of the 1,371 patients who were diagnosed with interstitial lung disease (ILD), 525 patients were selected to form the second dataset with the criteria of having computed tomography (CT) data from two different timepoints that are 40 weeks apart. In particular, the analysis engine 110 was deployed to predict, for each patient, all-cause survival and cause-specific (e.g., ILD-related) survival. In this context, survival prediction refers to the estimation of time to an event. For all-cause survival prediction, the event is death of all causes. In the case of cause-specific (e.g., ILD-related) survival prediction, the event is any death that is related to interstitial lung disease.

The performance of the analysis engine 110 was compared to three conventional methodologies. For lung cancer risk prediction, the performance of the analysis engine 110 was compared to that of a time-aware transformer, which assumes feature importance to be linearly decreasing over time. For survival prediction, the performance of the analysis engine 110 was compared with a 3D-ResNet-based convolutional neural network that has been used for all-cause survival prediction of idiopathic pulmonary fibrosis.

Table 1 below depicts a comparison of the area under the curve (AUC), which measures of the ability of a classifier to distinguish between classes, for lung cancer risk prediction in the third year using imaging data (e.g., computed tomography (CT) scans) from the first year and the second year.

TABLE 1

| Model name | AUC | Std |
|---|---|---|
| Generative deformable transformer | 0.61 | 0.03 |
| Time-distance transformer | 0.59 | 0.04 |

Table 2 below depicts a comparison of the concordance index, which measures the proportion of correct observations, for survival prediction (e.g., all-cause mortality and idiopathic pulmonary fibrosis specific mortality) for patients with idiopathic pulmonary fibrosis.

TABLE 2

| | | C-Index | | | |
|---|---|---|---|---|---|
| | Time | of | | C-Index of | |
| | points | all-cause | | ILD-related | |
| Methods | for input | survival | Std | survival | Std |
| Generative | Prior and | 0.63 | 0.02 | 0.64 | 0.01 |
| deformable | current | | | | |
| transformer | | | | | |
| 3D-ResNet-based | Current | 0.55 | 0.05 | 0.67 | 0.04 |
| convolutional neural | | | | | |
| network | | | | | |

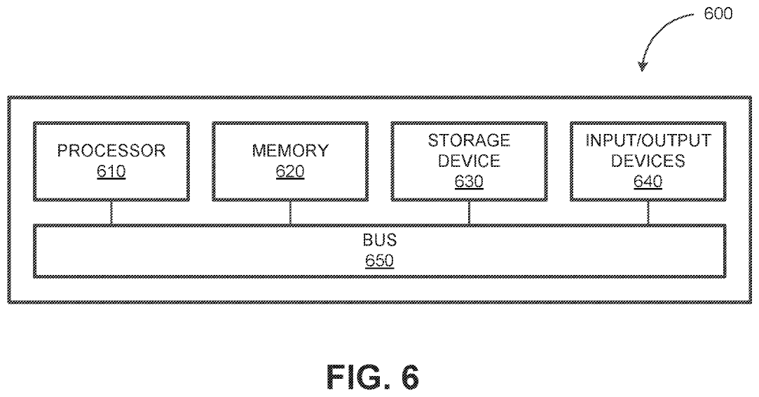

FIG. 6 depicts a block diagram illustrating an example of a computing system 600 consistent with implementations of the current subject matter. Referring to FIGS. 1-6, the computing system 600 can be used to implement the analysis engine 110, the client device 120, the data store 130, and/or any components therein.

As shown in FIG. 6, the computing system 600 can include a processor 610, a memory 620, a storage device 630, and an input/output device 640. The processor 610, the memory 620, the storage device 630, and the input/output device 640 can be interconnected via a system bus 650. The processor 610 is capable of processing instructions for execution within the computing system 600. Such executed instructions can implement one or more components of, for example, the analysis engine 110, the client device 120, the data store 130, and/or the like. In some example embodiments, the processor 610 can be a single-threaded processor. Alternately, the processor 610 can be a multi-threaded processor. The processor 610 is capable of processing instructions stored in the memory 620 and/or on the storage device 630 to display graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a solid state drive, a floppy disk device, a hard disk device, an optical disk device, or a tape device, or other suitable persistent storage means. The input/output device 640 provides input/output operations for the computing system 600. In some example embodiments, the input/output device 640 includes a keyboard and/or pointing device. In various implementations, the input/output device 640 includes a display unit for displaying graphical user interfaces.

According to some example embodiments, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some example embodiments, the computing system 600 can be used to execute various interactive computer software applications that can be used for organization, analysis and/or storage of data in various formats. Alternatively, the computing system 600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
training a generative transformer model to generate, based at least on a first training image from a first timepoint, a synthetic latent representation of a second training image from a second timepoint that enables the second training image to be reconstructed from the synthetic latent representation of the second training image;
applying the trained generative transformer model to generate, based at least on an input medical image, a synthetic latent representation for a different timepoint than a timepoint of the input medical image; and
determining, based at least on the synthetic latent representation for the different timepoint, a clinical assessment for the different timepoint.

2. (canceled)

3. (canceled)

4. The system of claim 1, wherein the generative transformer model includes an encoder trained to generate a latent representation of a medical image by at least extracting, from the medical image, one or more latent features, and wherein the generative transformer model further includes a decoder trained to reconstruct the medical image from the one or more latent features.

5. The system of claim 4, wherein the generative transformer model includes a longitudinal deformable attention mechanism having a flexible range of self-attention across a plurality of pixels comprising a medical image.

6. (canceled)

7. The system of claim 5, wherein the training of the generative transformer model includes
training the encoder to generate the first latent representation of the first training image that enables the decoder to reconstruct, from the first latent representation, the first training image.

8. The system of claim 7, wherein the training of the generative transformer model further includes training the longitudinal deformable attention mechanism to
determine, based at least on the first latent representation of the first training image, one or more offsets to shift feature extraction to one or more regions of the first training image that differentiate the first training image from the second training image,
generate, based at least on the one or more regions of the first training image, the synthetic latent representation of the second training image, and
generate the first synthetic latent representation of the second training image to enable the decoder to reconstruct, from the synthetic latent representation of the second training image, the second training image.

9. (canceled)

10. The system of claim 7, wherein the training of the generative transformer model includes
applying the encoder to generate a latent representation of the second training image, and
reducing a loss associated with a difference between the synthetic latent representation of the second training image and the latent representation of the second training image.

11. The system of claim 7, wherein the training of the generative transformer model includes
applying the decoder to reconstruct the second training image from the synthetic latent representation of the second training image, and
reducing a loss associated with a difference between the second training image and the second training image reconstructed from the synthetic latent representation of the second training image.

12. (canceled)

13. The system of claim 5, wherein the longitudinal deformable attention mechanism includes a plurality of weight matrices, and wherein the training of the generative transformer model includes adjusting one or more weights included in the plurality of weight matrices.

14. The system of claim 13, wherein the plurality of weight matrices include a query matrix representative of a focus patch, a key matrix creating a plurality of key vectors measuring a relevance or similarity between the focus patch and other patches in the input medical image, and a value matrix generating value vectors comprising contextual information for each patch associated with the input medical image.

15. The system of claim 14, wherein the training of the generative transformer model includes determining, based at least on the query matrix, an offset to shift feature extraction to one or more regions of the first training image having a threshold likelihood of exhibiting a clinically significant change between the first timepoint of the first training image and the second timepoint of the second training image.

16. The system of claim 15, wherein the offset is applied to the key matrix and the value matrix in order to shift feature extraction to the one or more regions of the first training image having a threshold likelihood of exhibiting a clinically significant change between the first timepoint of the first training image and the second timepoint of the second training image.

17. The system of claim 1, further comprising:
applying a clinical assessment computation model to determine, based at least on the synthetic latent representation for the different timepoint, the clinical assessment for the different timepoint.

18. The system of claim 17, further comprising:
training, based at least on training data, the clinical assessment computation model, the training data including the synthetic representation of the second training image, and the training of the clinical assessment computation model includes applying the clinical assessment computation model to determine, based at least on the synthetic representation of the second training image, a clinical assessment for the second timepoint of the second training image.

19. The system of claim 18, wherein the training of the clinical assessment computation model further includes reducing a loss associated with a difference between the clinical assessment for the second timepoint of the second training image and a ground-truth clinical assessment for the second timepoint of the second training image.

20. (canceled)

21. The system of claim 1, wherein the clinical assessment includes one or more of (i) a risk for a disease developing or recurring at the different timepoint, (ii) a progression of a disease at the different timepoint, and (iii) a survival prediction for a disease at the different timepoint.

22. (canceled)

23. (canceled)

24. The system of claim 1, wherein each of the first training image, the second training image, and the input medical image comprises one or more of a whole slide image (WSI), a computed tomography (CT) scan, a positron emission tomography (PET) scan, an X-ray, a magnetic resonance imaging (MRI) scan, and an ultrasound scan.

25. The system of claim 1, wherein the synthetic latent representation for the different timepoint corresponds to an unavailable medical image from the different timepoint, and wherein the trained generative transformer model generates the synthetic latent representation for the different timepoint absent the unavailable medical image.

26. (canceled)

27. (canceled)

28. The system of claim 1, wherein the first synthetic latent representation includes one or more latent features extracted from one or more regions of the first training image more likely to exhibit changes between the first training image and the second training image.

29. The system of claim 28, wherein each latent feature of the one or more latent features comprise a hidden feature determined based on one or more observable features in the first training image, and wherein the one or more observable features include an intensity value of one or more pixels in the first training image.

30. (canceled)

31. A computer-implemented method, comprising:
training a generative transformer model to generate, based at least on a first training image from a first timepoint, a synthetic latent representation of a second training image from a second timepoint that enables the second training image to be reconstructed from the synthetic latent representation of the second training image;
applying the trained generative transformer model to generate, based at least on an input medical image, a synthetic latent representation for a different timepoint than a timepoint of the input medical image; and
determining, based at least on the synthetic latent representation for the different timepoint, a clinical assessment for the different timepoint.

32. (canceled)
